(19) United States
(12) Patent Application Publication
(10) Pub. No.: US 2016/0022337 A1
(43) Pub. Date: Jan. 28, 2016

(54) COMPRESSION CLIP FOR FRACTURED BONE

(71) Applicant: EMPIRE TECHNOLOGY DEVELOPMENT LLC, Wilmington, DE (US)

(72) Inventor: Debasish PRADHAN, Sambalpur District (IN)

(21) Appl. No.: 14/809,793

(22) Filed: Jul. 27, 2015

(30) Foreign Application Priority Data
Jul. 26, 2014 (IN) 2120/DEL/2014

(51) Int. Cl. A61B 17/84

(52) U.S. Cl. CPC A61B 17/844; A61B 2017/681

(57) ABSTRACT

Methods and devices for fixation of a lateral compression fracture are disclosed. At least one compression device may be provided in a deployer. The deployer may be positioned adjacent to a facture line of a fractured bone. The at least one compression device may be laparoscopically delivered from the deployer to the fractured bone and secured the fractured bone. A compression device may include a central portion and a plurality of arms. The central portion may have a first side surface configured to contact a bone fracture and a second side surface. Each arm may extend from a proximal end attached to the central portion and have a distal end that includes a locking mechanism. Each locking mechanism may include at least one of a conical projection, a barb hook, a claw hook, and a serrated hook.

### CROSS-REFERENCE TO RELATED APPLICATION

This application claims priority benefit under Title 35 §119(a) to India Patent Application No. 2120/DEL/2014, filed Jul. 26, 2014, entitled “Compression Clip for a Fractured Bone,” the content of which is herein incorporated by reference in its entirety.

### BACKGROUND

The pelvic ring includes right and left ilium bones, the sacrum and their associated ligamentous connections. The main connections are through and around the right and left sacroiliac joints at the posterior of the pelvis and the pubic symphysis at the anterior of the pelvis. Anterior pelvic stability is provided by the pubic symphysis, the midline articulation of the pubic bones. At the pubic symphysis, the opposing bony surfaces are covered by cartilage and united by layers of fibrocartilage and fibrous tissue. The pubic symphysis is the weakest link in the pelvic ring, supplying only 15% of inherent pelvic stability.

The pelvic ring is a key structural element of the skeletal system because it is a weight-bearing structure interposed between the upper body and the legs. As such, if a fracture occurs and is left untreated, the pelvic ring may not heal (nonunion) or may heal in a poor position (malunion). Nonunion can lead to chronic pain and an inability to walk. Malunion can result in a short leg or a leg which points in the wrong direction. Because of these problems, it is necessary to reposition the fragments which have become displaced during the fracturing to normal (reduction). Once the fragments are repositioned, it is necessary to hold them in place (stabilization) until the healing of the fracture is complete. This process may take approximately 6 to 8 weeks.

Numerous different types of pelvic fractures may occur of which lateral compression (LC) fractures comprise one category. LC fractures are divided into three types: LC Type I (typically caused by a lateral force compressing the sacrum resulting in fractures of the ilium near the pubic symphysis and the sacrum), LC Type II (typically caused by a lateral force compressing an ilium resulting in fractures of the ilium near the sacroiliac joint and the pubis symphysis), and LC Type III (an LC Type I/II fracture and a contralateral anterior posterior compression (APC) typically resulting from a rollover vehicle accident or being trapped between unyielding objects).

### SUMMARY

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

In an embodiment, a method for fixation of a lateral compression fracture may include providing at least one compression device in a deployer, positioning the deployer adjacent to a fracture line of a fractured bone, laparoscopically delivering the at least one deployer to the fractured bone, and securing the at least one compression device to the fractured bone.

In an embodiment, a method of fixation of a lateral compression fracture may include attaching at least one compression device to a fractured bone using at least one attachment device. The at least one compression device may be configured to have a central portion with a plurality of arms. Each arm may have a locking mechanism comprising at least one of a conical projection, a barb hook, a claw hook, and a serrated hook.

In an embodiment, a compression device for fixation of a lateral compression fracture may include a central portion having a first side surface and a second side surface and a plurality of arms. The first side surface may be configured to contract a bone fracture. Each arm may extend from the central portion, have a proximal end attached to the central portion, and have a distal end comprising a locking mechanism. The locking mechanism of each of the plurality of arms may comprise at least one of a conical projection, a barb hook, a claw hook, and a serrated hook.

### BRIEF DESCRIPTION OF THE FIGURES

FIG. 1A depicts a schematic of an illustrative lateral compression device in a pre-formed condition according to an embodiment.

FIG. 1B depicts a schematic of an illustrative lateral compression device in an expanded condition according to an embodiment.

FIG. 2 depicts a schematic of an illustrative lateral compression device according to an alternate embodiment.

FIG. 3 depicts a schematic of an illustrative lateral compression device according to yet another alternate embodiment.

FIGS. 4A-C depict schematics of illustrative locking mechanisms for a lateral compression device according to embodiments.

FIG. 5 depicts a flow diagram of an illustrative method for fixation of a lateral compression device according to an embodiment.

FIG. 6 depicts a flow diagram of an illustrative method for fixation of a lateral compression fracture according to an embodiment.

### DETAILED DESCRIPTION

The following term shall have, for the purposes of this application, the meaning set forth below:

A “deployer” refers to a device used to position one or more compression devices. The deployer may be an instrument used to deliver at least one compression device laparoscopically. The deployer may be used to store at least one compression device in a pre-formed condition within a shaft of the deployer.

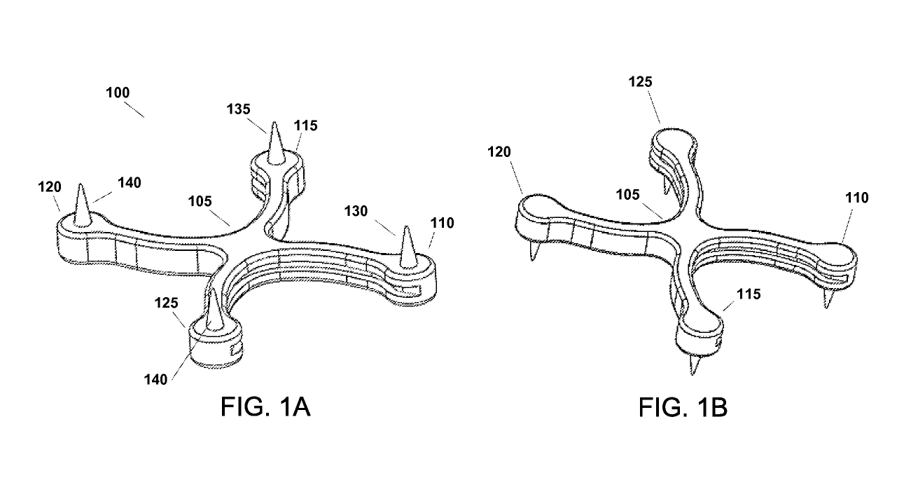

FIGS. 1A, 1B, 2 and 3 depict schematics of illustrative lateral compression devices according to various embodiments. As shown in FIG. 1A, a lateral compression device 100 in a pre-formed condition may include a central portion 105 and a plurality of arms, such as 110-125. Each arm 110-125 may include a corresponding locking mechanism, such as 130-145, respectively.

In an embodiment, the central portion 105 may have a first side surface and a second side surface. The first side surface and the second side surface may be on opposing sides of the central portion 105 of the lateral compression device 100. The first side surface of the central portion 105 may be configured to contact a bone fracture.

A lateral compression device may include a plurality of arms, such as two arms, three arms, four arms, or the like. Each arm 110-125 may extend from the central portion 105 such that a proximal end is attached to the central portion and a distal end comprises a corresponding locking mechanism 130-145.

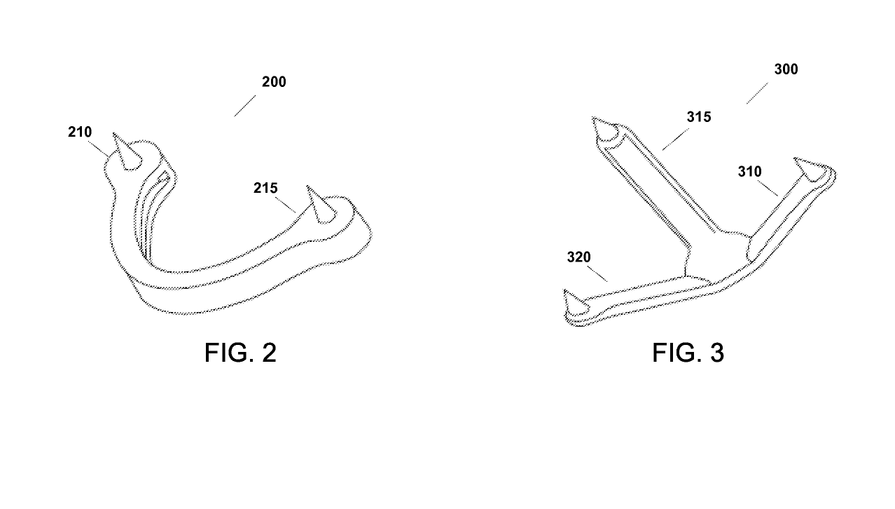

In some embodiments, as depicted in FIGS. 1A and 1B, the lateral compression device 100 may have four arms 110-125 and may be substantially X-shaped. In some embodiments, such as is depicted in FIG. 2, a lateral compression device 200 may have a U-shape with two arms 210, 215. In some embodiments, such as is depicted in FIG. 3, a lateral compression device 300 may have three arms 310, 315, 320 substantially in a T-shape or a Y-shape.

In some embodiments, the central portion 105 may be substantially rigid and ductile (i.e., have a relatively low stiffness), and each arm 110-125 may be substantially flexible with an area of high stiffness surrounding the corresponding locking mechanism 130-145. In an embodiment, the lateral compression device 100 may be formed from a functionally graded material sheet that is made of a plurality of sections having different stiffness values. In such an embodiment, the lateral compression device 100 may be cut from the functionally graded material with sections corresponding to ductility required for each portion of the lateral compression device. In other embodiments, various stiffness values may be achieved by quantum of work hardening as per the property required for each portion of the lateral compression device 100. In yet other embodiments, the central portion 105 may be made of a first material having a relatively low stiffness, and the arms 110-125 may be made of a second dissimilar material having a relatively high stiffness. In still other embodiments, one or more stiffening members (not shown) may be located within a base material. A first stiffening member may be a curved spring element that extends from a first arm, such as 110, into the central portion 105 and out to a second arm, such as 115. A second stiffening member may be a corresponding curved spring element that extends from a third arm, such as 120, into the central portion 105 and out to a fourth arm, such as 125.

A lateral compression device 100 may be made of any material that is commonly used for implanted orthopedic devices. For example, a lateral compression device 100 may comprise stainless steel, a stainless steel alloy, titanium, a titanium alloy, a polymer, or any combination thereof. In particular, stainless steel alloys may be used for portions of the lateral compression device 100 that require strength, flexibility and/or biocompatibility. Titanium may be used to provide a surface on which bone grows and strongly adheres because its surface quickly oxidizes upon exposure to air. In an embodiment, the compression device may comprise a bioabsorbable polymer, such as poly(glycolic acid), poly(L-lactic acid), poly(ortho ester), poly(epsilon-caprolactone), poly(hydroxybutyrate valerate) or any combination thereof.

The arms 110-125 of a lateral compression device 100 may be made of a material having limited flexibility. In an embodiment, the arms 110-125 may be made in a pre-formed condition, such as is shown in FIG. 1A. The arms 110-125 may be bent during application to approximate the contour of the bone into which the lateral compression device 100 is inserted.

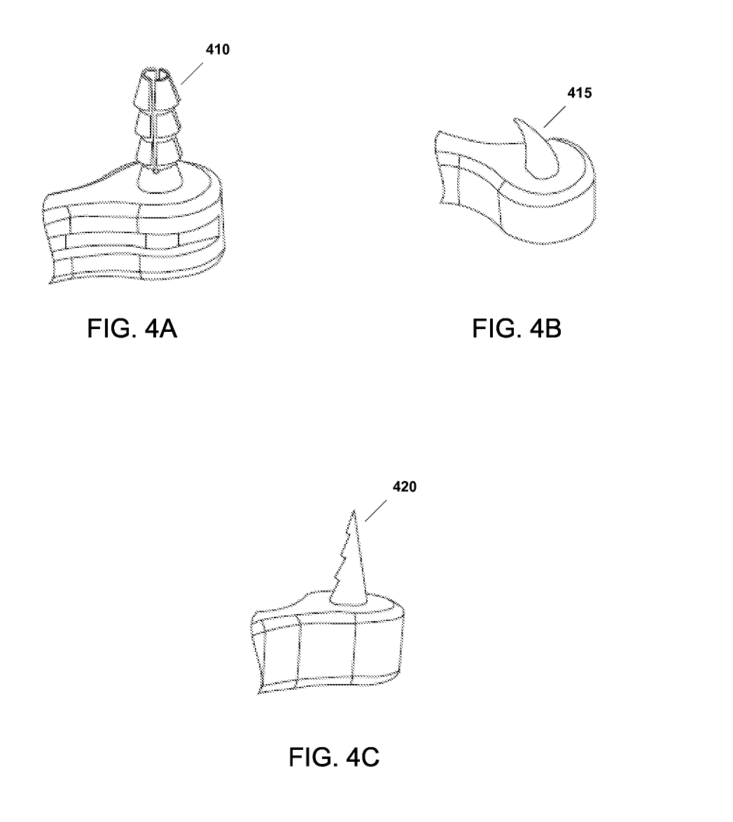

FIG. 1A and FIGS. 4A-D depict schematics of illustrative locking mechanisms for a lateral compression device according to embodiments. In some embodiments, a locking mechanism, such as 130-145 in FIG. 1A, may include a conical projection having a sharp tip. The locking mechanism 130-145 may pierce into the bone surface and rigidly lock the clip onto the bone.

In alternate embodiments, a locking mechanism, such as 410 in FIG. 4A, may include a barb hook. The locking mechanism 410 may include one or more vertical slits that enable the locking mechanism to compress inwards during insertion into a bone. Upon insertion, the outer rack shape of the locking mechanism 410 may provide secure fixation within the bone and prevent slippage.

In still other embodiments, a locking mechanism, such as 415 in FIG. 4B, may include a claw hook that pierces into a bone angularly. The curved claw hook locking mechanism 415 may be configured to hold in place securely and rigidly without releasing from the site of the fracture.

In yet other embodiments, a locking mechanism, such as 420 in FIG. 4C, may include a serrated hook. The locking mechanism 420 may have a sharp conical projection. The rack shape of the serrated hook locking mechanism 420 may prevent slipping of the mechanism when pierced into bone, thereby providing secure locking of the mechanism.

In alternate embodiments, a locking mechanism may include any other fastening mechanism including, without limitation, a removable screw. In such embodiments, the arms of a lateral compression device may include holes through which the screws are threaded into the bone. In an embodiment, the removable screw locking mechanism may comprise a self-tapping screw that is guided and fixed on the bone surface. Screw locking may provide secure and rigid locking of the lateral compression device.

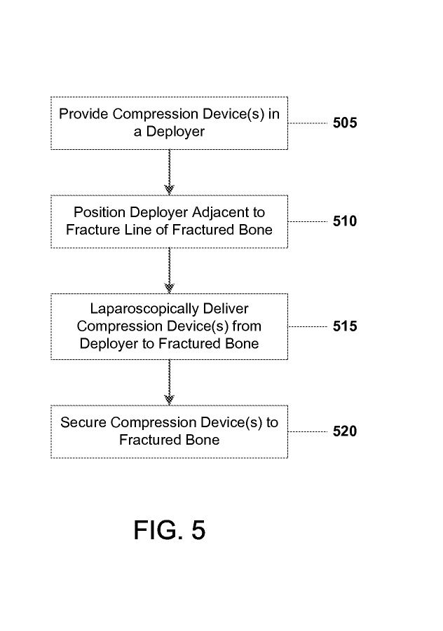

FIG. 5 depicts a flow diagram of an illustrative method for fixation of a lateral compression device according to an embodiment. As shown in FIG. 5, at least one compression device may be provided 505 in a deployer. In some embodiments, the compression device may be provided 505 in the deployer in a pre-formed condition, such as is shown in FIG. 1A. In the pre-formed condition, arms of the compression device may be in a relaxed or narrow position.

The deployer may be positioned 510 adjacent to a fracture line of a fractured bone. In some embodiments, the fractured bone may be a fractured pelvic bone, such as the ilium, the pubic symphysis or the sacrum. In some embodiments, the fractured bone may be reduced before the deployer is positioned 510. In other embodiments, the fractured bone may be reduced while the deployer is positioned 510. In still other embodiments, the fractured bone may be reduced after the deployer is positioned 510.

The at least one compression device may be laparoscopically delivered 515 from the deployer to the fractured bone. In an alternate embodiment, the at least one compression device may be delivered 515 through an open surgical procedure. In an embodiment, the at least one compression device may be laparoscopically delivered 515 in a completely expanded condition, such that the position of the arms of the at least one compression device are spread wider than in the pre-formed condition.

In an embodiment, the at least one compression device may be formed into a completely expanded condition as a result of its traversal through the deployer. As the compression device is delivered 515, a distal compression device may be advanced through a shaft of the deployer. The shaft may have an internal profile that causes the arms of the compression device to be repositioned into a completely expanded condition.

In some embodiments, the at least one compression device may include a plurality of compression devices. In some embodiments in which the at least one compression device includes a plurality of compression devices, each of the plurality of compression devices may be delivered 515 at a different time, to a different position on the fractured bone, and/or at a different orientation on the fractured bone.

In some embodiments, a plurality of fixation holes may be formed on a first side and a second side of a fracture line of the fractured bone. In other words, fixation holes may be formed such that at least one hole is located on either side of the fracture line in the fractured bone. The holes may be used to align the deployer during positioning 510 so that the hooking mechanisms of the compression device are seated in the holes upon delivery 515. As such, the at least one locking mechanism of the at least one compression device may be inserted into at least one fixation hole on the fractured bone as a result of the delivery 515 of the compression device.

The at least one compression device may be secured 520 to the fractured bone upon delivery 515. In some embodiments, a compression device may be secured 520 as a result of the locking mechanisms of the compression device being delivered 515 into the corresponding fixation holes and being released from the deployer in a completely expanded position. In such embodiments, the locking mechanisms of the compression device may be seated in the fixation holes and may exert a compressive force as a result of the compression device trying to return to the relaxed position. As a result, a dynamic compression force may be applied along the fractured bone after securing 520 the at least one compression device to the fractured bone.

In other embodiments, such as for compression devices using removable screws as locking mechanisms, the compression device may be positioned across the fracture line of the fractured bone and held in an expanded position while affixing the screws through the arms of the compression device. Upon placement of the screws, the compression device may be secured 520 and exert a compressive force in the manner described above.

As such, upon delivery 515 and securing 520 of the at least one compression device into the fractured bone in a completely expanded state, internal stresses generated in the at least one compression device may generate a compressive force along the fracture and result in a rigid dynamic compressive fixation. The at least one compression device may be manufactured in a variety of different sizes for delivery to a fracture site based on the type and severity of the fracture.

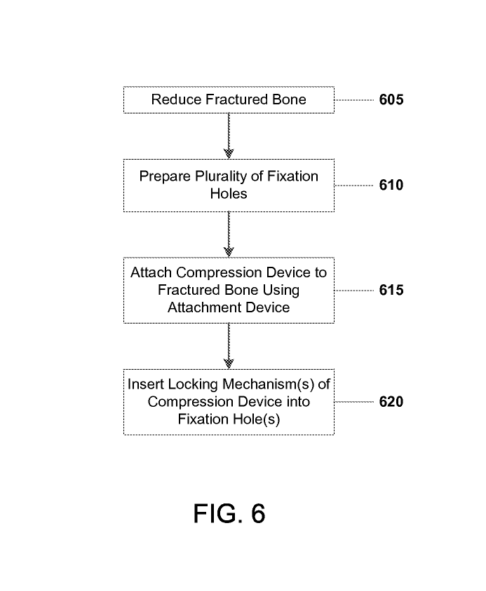

FIG. 6 depicts a flow diagram of an alternate illustrative method for fixation of a lateral compression fracture according to an embodiment. As shown in FIG. 6, at least one compression device may be attached 615 to a fractured bone using at least one attachment device. The at least one compression device is configured to have a central portion with a plurality of arms. Each arm has a locking mechanism. Each locking mechanism may include at least one of a conical projection, a barb hook, a claw hook, and a serrated hook. The attachment device may be a deployer configured to deliver the at least one compression device to the lateral compression fracture. In some embodiments, each of the plurality of arms of the at least one compression device may be placed in an expanded condition using the attachment device.

In some embodiments, the fractured bone may be reduced 605 before the at least one compression device is attached 615 to the fractured bone. In other embodiments, the fractured bone may be reduced 605 during the time that the at least one compression device is attached 615 to the fractured bone. In yet other embodiments, the fractured bone may be reduced 605 after the at least one compression device is attached 615 to the fractured bone. In some cases, the fractured bone may be a fractured pelvic bone.

In some embodiments, a plurality of fixation holes may be prepared 610. In some embodiments, a fixation hole may be prepared 610 before a compression device is attached 615 to the fractured bone. In some embodiments, a fixation hole may be prepared 610 during the attachment 615 of a compression device to the fractured bone. In some embodiments, the fixation hole may be prepared 610 as a result of the attachment 615 of a compression device to the fractured bone. Fixation holes may be prepared 610 on a first side of the fractured bone and on a second side of the fractured bone. For example, the first side of the fractured bone may be on a first side of the lateral compression fracture, and the second side of the fractured bone may be on a second opposing side of the lateral compression fracture. The plurality of fixation holes may be prepared 610 using a template. The particular template used to prepare 610 the plurality of fixation holes may be selected based on the size of at least one compression device to be used to fix the fractured bone.

In some embodiments, at least one locking mechanism of the at least one compression device may be inserted 620 into at least one fixation hole on the fractured bone. The at least one locking mechanism may be used to fix the at least one compression device around a fracture of the fractured bone.

In some embodiments, one or more compression devices may be used to apply a compressive force to assist in keeping the fractured bone fixed in place. For example, when a compression device is formed into an expanded condition, the compression device may naturally seek to return to the pre-formed condition because of, for example, a stiffening member. As such, when a compression device is placed across a fracture in a fractured bone, the tendency of the stiffening member to try to return to the compression device to its pre-formed condition is used to apply a compressive force that keeps the fractured bone from becoming unfixed.

Although the devices and methods described above have been discussed in reference to fractures in pelvic bones, it will be apparent to those of skill in the art that the device may also be used to fix fractures occurring in other boney structures.

### Example

A plurality of X-shaped compression clips are loaded into a laparoscopic instrument for deployment. The laparoscopic instrument has a shaft which receives the compression clips at a proximal end and a plunger used to engage the compression clip that is most distal from the point at which the compression clips are inserted. The shaft is shaped such that the stored compression clips are stored in a relaxed condition, and a compression clip that is to be deployed is in an expanded condition.

A bone fracture is reduced using any conventional method. Two holes are formed on each side of the fracture using a template that is sized based on the compression clip to be deployed.

The fractured bone is kept in the reduced condition as the shaft of the laparoscopic instrument is aligned to the holes made in the bone. The laparoscopic instrument is activated causing the plunger to engage the most distal compression clip and advance the compression clip through the shaft towards the bone. As the plunger advances past the distal end of the laparoscopic instrument, the compression clip is deployed such that the compression clip is inserted into the holes in the bone in a completely expanded condition. Because the compression clip is in an expanded condition, the clip tries to return to its pre-formed condition. As a result, the compression clip applies a dynamic compression force along the fractured resulting in rigid fixation.

The plunger of the laparoscopic instrument retracts back to its original position to be made ready to engage a next compression clip.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially” of or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera As a non-limiting example, each range discussed herein can be readily broken down into a lower third, middle third and upper third, et cetera As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A method for fixation of a lateral compression fracture, the method comprising:
providing at least one compression device in a deployer;
positioning the deployer adjacent to a fracture line of a fractured bone;
laparoscopically delivering the at least one compression device from the deployer to the fractured bone; and
securing the at least one compression device to the fractured bone.

2. The method of claim 1, further comprising:
preparing a plurality of fixation holes on a first side and a second side of the fractured bone; and
inserting at least one locking mechanism of the at least one compression device into at least one fixation hole on the fractured bone.

3. The method of claim 1, wherein providing at least one compression device comprises providing the at least one compression device in the deployer in a pre-formed condition.

4. The method of claim 1, wherein laparoscopically delivering the at least one compression device comprises laparoscopically delivering the at least one compression device in a completely expanded condition.

5. The method of claim 1, wherein:
providing at least one compression device comprises providing a plurality of compression devices, and
laparoscopically delivering the plurality of compression devices comprises delivering each of the plurality of compression devices at a different time.

6. The method of claim 1, wherein:
providing at least one compression device comprises providing a plurality of compression devices, and
laparoscopically delivering the plurality of compression devices comprises delivering each of the plurality of compression devices to a different position on the fractured bone.

7. The method of claim 1, wherein:
providing at least one compression device comprises providing a plurality of compression devices, and
laparoscopically delivering the plurality of compression devices comprises delivering each of the plurality of compression devices at a different orientation on the fractured bone.

8. The method of claim 1, wherein the at least one compression device is configured to have a central portion comprising a plurality of arms, wherein each arm comprises a locking mechanism.

9. A method for fixation of a lateral compression fracture, the method comprising:
attaching at least one compression device to a fractured bone using at least one attachment device, wherein the at least one compression device is configured to have a central portion with a plurality of arms, wherein each arm has a locking mechanism comprising at least one of a conical projection, a barb hook, a claw hook, and a serrated hook.

10. The method of claim 9, further comprising reducing the fractured bone.

11. The method of claim 9, further comprising:
preparing a plurality of fixation holes on a first side and a second side of the fractured bone; and
inserting at least one locking mechanism of the at least one compression device into at least one fixation hole on the fractured bone.

12. The method of claim 9, further comprising:
preparing a plurality of fixation holes on a first side and a second side of the fractured bone such that at least a first fixation hole is on one side of the fracture and at least a second fixation hole is on an opposing side of the fracture;
aligning the at least one compression device with the fractured bone such that at least one locking mechanism is inserted into the first fixation hole and at least one locking mechanism is inserted into the second fixation hole; and
applying a compressive force to the fractured bone by the at least one compression device.

13. The method of claim 9, wherein attaching the at least one compression device further comprises:
expanding the at least one compression device from an initial state into an expanded state, thereby resulting in an expanded compression device;
attaching the expanded compression device to the fractured bone; and
reducing the fractured bone as a result of internal stresses within the expanded compression device, wherein the internal stresses cause the expanded compression device to return to the initial state.

14. The method of claim 13, further comprising:
preparing a plurality of fixation holes on a first side and a second side of the fractured bone such that at least a first fixation hole is on one side of the fracture and at least a second fixation hole is on an opposing side of the fracture;
aligning the expanded compression device with the fractured bone such that at least one locking mechanism is inserted into the first fixation hole and at least one locking mechanism is inserted into the second fixation hole; and
applying a compressive force to the fractured bone by the expanded compression device.

15. A compression device for fixation of a lateral compression fracture comprising:
a central portion having a first side surface and a second side surface, wherein the first side surface is configured to contact a bone fracture; and
a plurality of arms, wherein each arm extends from the central portion, has a proximal end attached to the central portion and has a distal end comprising a locking mechanism,
wherein the locking mechanism of each of the plurality of arms comprises at least one of a conical projection, a barb hook, a claw hook, and a serrated hook.

16. The compression device of claim 15, wherein the compression device has a plurality of stiffness areas such that the central portion comprises a first stiffness area and each of the plurality of arms comprises a second stiffness area.

17. The compression device of claim 15, wherein each of the plurality of arms comprises:
a rigid portion adjacent the locking mechanism at the distal end; and
a flexible portion positioned between the proximal end and the rigid portion.

18. The compression device of claim 15 wherein the locking mechanism is configured to pierce into a bone and rigidly lock the compression device to the bone.

19. The compression device of claim 15, wherein the compression device comprises a stainless steel, a stainless steel alloy, titanium, a titanium alloy, a polymer, or a combination thereof.

20. The compression device of claim 15, wherein the compression device comprises a bioabsorbable polymer selected from the group comprising poly(glycolic acid), poly(L-lactic acid), poly(ortho ester), poly(epsilon-caprolactone), poly(hydroxybutyrate valerate), and any combination thereof.
